(19) United States
(12) Patent Application Publication
(10) Pub. No.: US 2009/0062736 A1
(43) Pub. Date: Mar. 5, 2009

(54) SAFETY SYRINGE ASSEMBLY

(76) Inventor: Lin-Li Jan, Taipei Hsien (TW)

Correspondence Address: Lin-Li Jan, 235 Chung - Ho, Box 8-24, Taipei, TW

(21) Appl. No.: 11/848,240

(22) Filed: Aug. 31, 2007

(51) Int. Cl. A61M 5/32

(52) U.S. Cl. 604/110; 604/197

(57) ABSTRACT

A safety syringe assembly comprises a syringe cover including: a slot being formed at a rear end of the syringe cover; a wing extending from a periphery of the slot; a buckle section being formed at an inner side of the syringe cover; the buckle section including an annular recess; a front side of the annular recess being greater than a rear side thereof; a syringe having a needle at a front end thereof and a hole at a rear end thereof; an outer periphery of the rear end of the syringe being formed with a flange; and a guide slope is formed in an inner wall of the syringe cover near a rear opening of the syringe cover for guiding an object inserted therethrough. The buckle section includes an annular recess; and a front side of the annular recess is greater than a rear side thereof.

### FIELD OF THE INVENTION

The present invention relates to syringes, and particularly to a safety syringe assembly, wherein as a syringe needle is placed in a syringe cover, the needle can be firmly secured in the syringe cover. The operation of the insertion and retracting needle from the syringe cover is easy.

### BACKGROUND OF THE INVENTION

There are many prior arts disclosed in the structure about syringe s, such as U.S. Pat. No. 7,247,151, in that an injector device comprises: a body having a syringe cavity and incorporating a syringe, the syringe comprising: a barrel defining a reservoir for an injectable medicament; an injection needle in communication with the reservoir such that the medicament content in the reservoir can be expelled from the reservoir via the needle; an internal piston which can be moved longitudinally only in an injecting direction along inside the barrel toward the needle to inject the medicament content through the needle, the needle having a point, and the syringe being slidably moveable within the syringe cavity in the body between a first configuration in which the needle has its point shielded, and a second configuration in which the needle is exposed for use for an injection to be administered; a plunger cavity; guide channels that extend along the sides of the plunger cavity; a plunger shaft located in the plunger cavity for moving the internal piston in the injecting direction, the plunger shaft having a head part with protrusions that are slidably received in the guide channels; and a snap-off cap that is attached to the body by a frangible link to close the plunger cavity and which must be removed to expose the head part of the plunger shaft prior to the injector device being used; wherein the plunger shaft is slidably moveable within the plunger cavity by a manual urging force applied to the head part by a user thereto toward the syringe to urge the syringe toward the second configuration and to urge the internal piston in the injecting direction; and wherein the syringe is biased to be returned toward its first configuration after the manual urging force has been removed.

However, most of the prior art syringe is difficult to operations or has a complicated structure, even the needle of the syringe can not be firmly secured. All these problems are necessary to be improved.

### SUMMARY OF THE INVENTION

Accordingly, the primary object of the present invention is to provide a safety syringe assembly, wherein as a syringe needle is placed in a syringe cover, the needle can be firmly secured in the syringe cover. The operation of the insertion and retracting needle from the syringe cover is easy.

To achieve above objects, the present invention provides a safety syringe assembly, comprising: a syringe cover including: a slot being formed at a rear end of the syringe cover; a wing extending from a periphery of the slot; a buckle section being formed at an inner side of the syringe cover; the buckle section including an annular recess; a front side of the annular recess being greater than a rear side thereof; a syringe having a needle at a front end thereof and a hole at a rear end thereof; an outer periphery of the rear end of the syringe being formed with a flange.

The wing has an approximate U shape. The wing has a greater upper end and a small lower end. The safety syringe assembly further comprises a guide slope is formed in an inner wall of the syringe cover near a rear opening of the syringe cover for guiding an object inserted therethrough. The buckle section includes an annular recess. A front side of the annular recess is greater than a rear side thereof. A push block has a projection at a front end thereof; and a rear end of the push block is expanded to have a size greater than the projection.

The various objects and advantages of the present invention will be more readily understood from the following detailed description when read in conjunction with the appended drawing.

### BRIEF DESCRIPTION OF THE DRAWINGS

FIG. 1 is a perspective view showing the syringe cover and syringe of the present invention.

FIG. 2 shows that the syringe is inserted into the syringe cover, and the push block is not engaged to the syringe cover.

FIG. 3 shows a longitudinal cross sectional view of FIG. 2.

FIGS. 4A to 4C show the release operation of the present invention.

FIG. 4A shows that the user's fingers hold the wing of the syringe cover, and the cylinder with an engaging head is to be inserted into the syringe cover.

FIG. 4B illustrates that the engaging end is inserted into the annular recess of the syringe cover and is then engaged with the syringe.

FIG. 4C illustrates that the syringe with the cylinder is taken out from the syringe cover.

FIGS. 5A to 5C shows the operation of engaging the syringe with the cylinder to the syringe cover.

FIG. 5A shows that the user holes the front end of the syringe cover, the cylinder with the syringe is placed at an upper side of the syringe cover.

FIG. 5A shows that the needle of the syringe passes through the slot of the syringe cover.

FIG. 5C shows that the syringe with the cylinder is pushed forwards so that the syringe is placed into the syringe cover and the cylinder is located at an opening of the syringe cover.

FIGS. 6A to 6C are cross sectional views showing the operation of engaging the syringe with the cylinder to the syringe cover.

FIG. 6A shows that the user holes the front end of the syringe cover, the cylinder with the syringe is placed at an upper side of the syringe cover.

FIG. 6B shows that the needle of the syringe passes through the slot of the syringe cover.

FIG. 6C shows that the syringe with the cylinder is pushed forwards so that the syringe is placed into the syringe cover and the cylinder is located at an opening of the syringe cover.

FIG. 6D shows that the cylinder is withdrew from the syringe cover.

FIGS. 7A to 7D show the operation of withdrawing the cylinder and inserting the push block.

FIG. 7A illustrates that the syringe with the cylinder is inserted into the syringe cover.

FIG. 7B illustrates that the cylinder is withdrew from the syringe cover.

FIG. 7C illustrates that the push block is to be inserted into the cylinder.

FIG. 7D illustrates that the push block is pushed into the syringe cover so as to seal the syringe into the syringe cover.

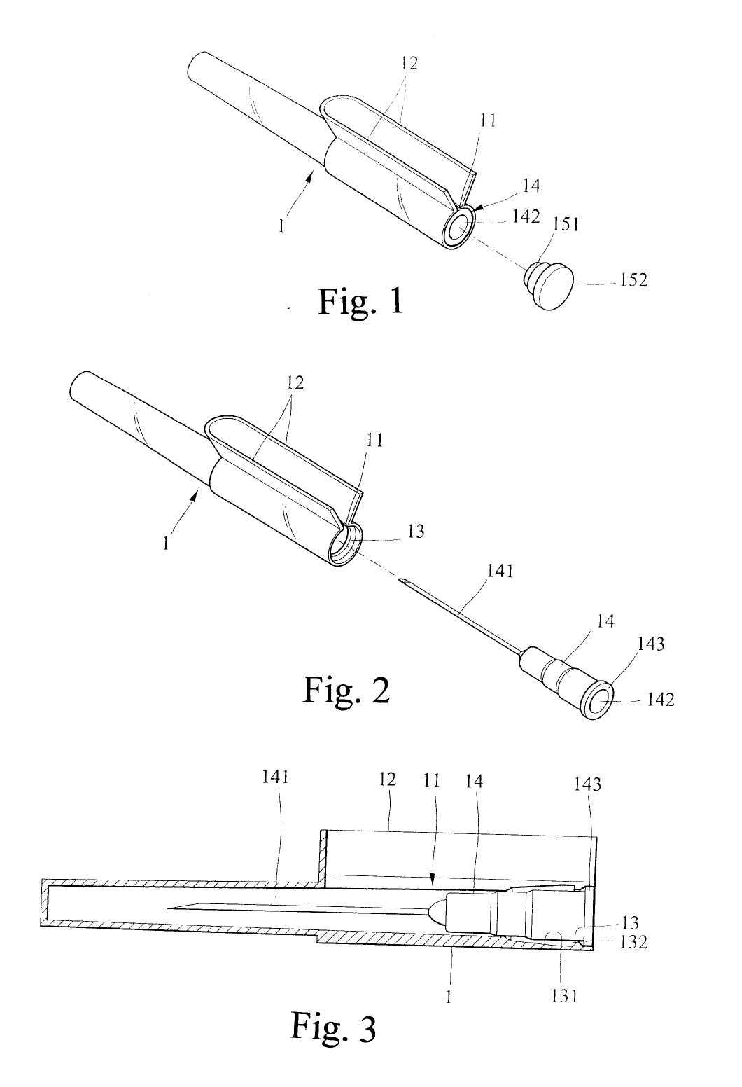

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

Referring to FIG. 3, the structure of the present invention is illustrated. The present invention has the following elements with reference to FIG. 2.

A syringe cover 1 includes the following elements.

A slot 11 is formed at a rear end of the syringe cover 1.

A wing 12 has an approximate U shape with a greater upper end and a small lower end. The lower end is connected to a periphery of the slot 11.

A buckle section 13 is formed at an inner side of the syringe cover 1. The buckle section 13 includes an annular recess 131. A front side of the annular recess 131 is greater than a rear side thereof.

A guide slope 132 is formed in an inner wall of the syringe cover 1 near a rear opening of the syringe cover 1 for guiding an object inserted therethrough.

A syringe 14 has a needle 141 at a front end thereof and a hole 142 at a rear end thereof. An outer periphery of the rear end of the syringe 14 is formed with a flange 143.

A push block 15 has a projection 151 at a front end thereof. A rear end of the push block 15 is expanded to have a size greater than the projection 151.

FIG. 2 shows that the syringe 14 is inserted into the syringe cover 1.

FIG. 3 shows a longitudinal cross sectional view of FIG. 2.

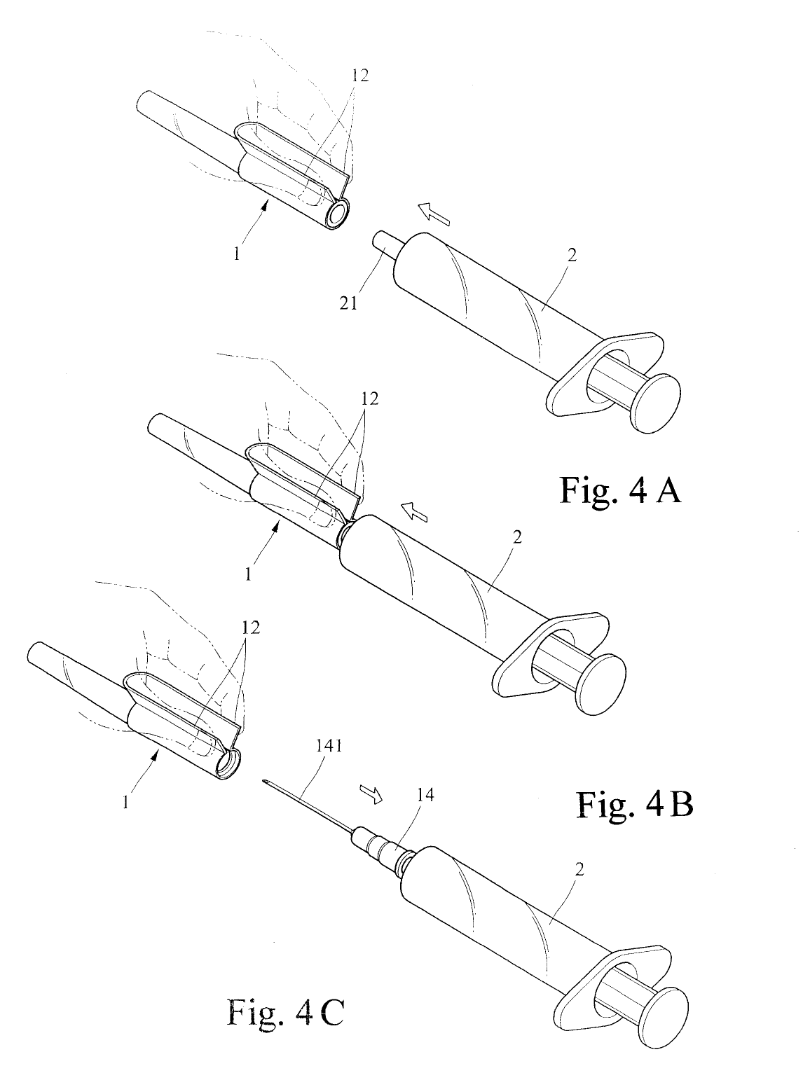

FIGS. 4A to 4C show the release operation of the present invention, in FIG. 4A, the user's fingers hold the wing 12 of the syringe cover 1, and the cylinder 2 with an engaging head is to be inserted into the syringe cover 1. In FIG. 4B, it is illustrated that the engaging end is inserted into the annular recess 131 of the syringe cover 1 and is then engaged with the syringe 14. In FIG. 4C, it is illustrated that the syringe 14 with the cylinder 2 is taken out from the syringe cover 1.

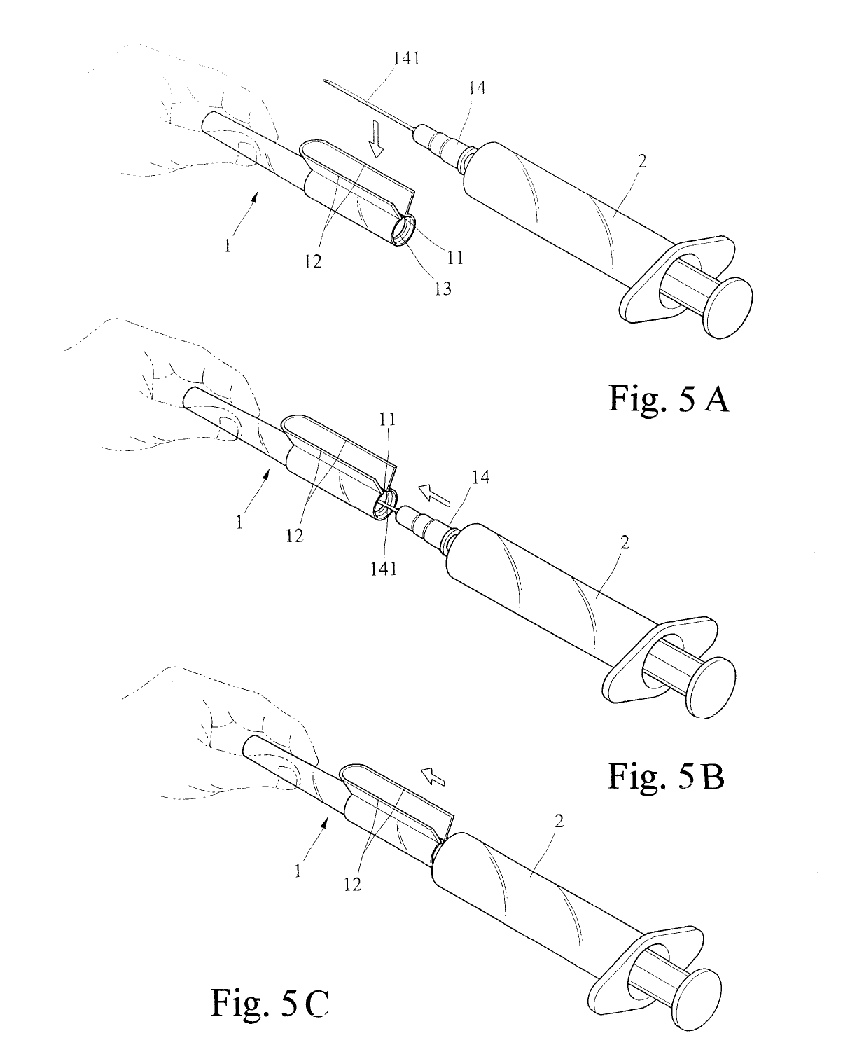

FIGS. 5A to 5C shows the operation of engaging the syringe 14 with the cylinder 2 to the syringe cover 1. In FIG. 5A, the user holes the front end of the syringe cover 1, the cylinder 2 with the syringe 14 is placed at an upper side of the syringe cover 1. In FIG. 5A, the needle 141 of the syringe 14 passes through the slot 11 of the syringe cover 1. In FIG. 5C, the syringe 14 with the cylinder 2 is pushed forwards so that the syringe 14 is placed into the syringe cover 1 and the cylinder 2 is located at an opening of the syringe cover 1.

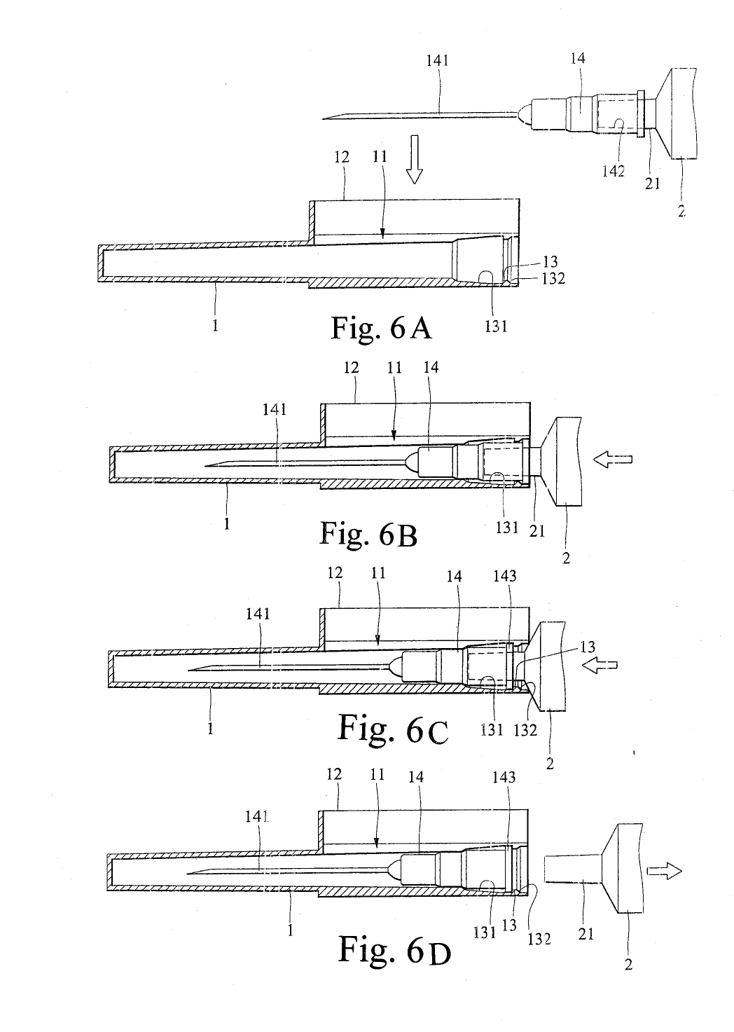

FIGS. 6A to 6C are cross sectional views showing the operation of engaging the syringe 14 with the cylinder 2 to the syringe cover 1. In FIG. 6A, the user holes the front end of the syringe cover 1, the cylinder 2 with the syringe 14 is placed at an upper side of the syringe cover 1. In FIG. 6B, the needle 141 of the syringe 14 passes through the slot 11 of the syringe cover 1. In FIG. 6C, the syringe 14 with the cylinder 2 is pushed forwards so that the syringe 14 is placed into the syringe cover 1 and the cylinder 2 is located at an opening of the syringe cover 1. In FIG. 6D, the cylinder 2 is withdrew from the syringe cover 1.

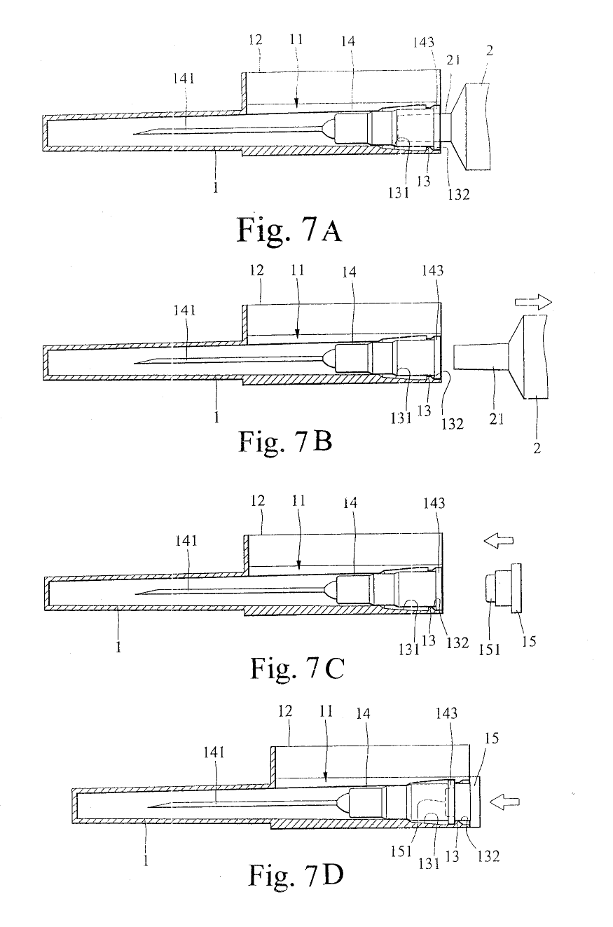

FIGS. 7A to 7D show the operation of withdrawing the cylinder 2 and inserting the push block 15. In FIG. 7A, it is illustrated that the syringe 14 with the cylinder 2 is inserted into the syringe cover 1. In FIG. 7B, it is illustrated that the cylinder 2 is withdrew from the syringe cover 1. In FIG. 7C, it is illustrated that the push block 15 is to be inserted into the cylinder 2. In FIG. 7D, it is illustrated that the push block 15 is pushed into the syringe cover 1 so as to seal the syringe 14 into the syringe cover 1.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A safety syringe assembly, comprising:
a syringe cover including:
a slot being formed at a rear end of the syringe cover;
a wing extending from a periphery of the slot;
a buckle section being formed at an inner side of the syringe cover; the buckle section including an annular recess; a front side of the annular recess being greater than a rear side thereof;
a syringe having a needle at a front end thereof and a hole at a rear end thereof; an outer periphery of the rear end of the syringe being formed with a flange.

2. The safety syringe assembly as claimed in claim 1, wherein the wing has an approximate U shape.

3. The safety syringe assembly as claimed in claim 1, wherein the wing has a greater upper end and a small lower end.

4. The safety syringe assembly as claimed in claim 1, further comprising: a guide slope is formed in an inner wall of the syringe cover near a rear opening of the syringe cover for guiding an object inserted therethrough.

5. The safety syringe assembly as claimed in claim 1, wherein the buckle section includes an annular recess; a front side of the annular recess is greater than a rear side thereof.

6. The safety syringe assembly as claimed in claim 1, wherein a push block has a projection at a front end thereof; and a rear end of the push block is expanded to have a size greater than the projection.
